(19) United States
(12) Patent Application Publication
(10) Pub. No.: US 2026/0000293 A1
(43) Pub. Date: Jan. 1, 2026

(54) Methods for Improving Macular Pigment Optical Density Measurement by Accounting for Melanin Absorption Using Heterochromatic Flicker Photometry (HFP)

(71) Applicant: Mohsen Sharifzadeh, Cottonwood Heights, UT (US)

(72) Inventor: Mohsen Sharifzadeh, Cottonwood Heights, UT (US)

(21) Appl. No.: 18/757,456

(22) Filed: Jun. 27, 2024

(51) Int. Cl. A61B 3/12; A61B 3/00; A61B 3/14

(52) U.S. Cl. CPC A61B 3/12; A61B 3/0025; A61B 3/14

(57) ABSTRACT

The present invention relates to methods for measuring macular pigment optical density (MPOD) more accurately by accounting for melanin absorption. Traditional heterochromatic flicker photometry (HFP) techniques do not consider melanin, leading to inaccuracies. This patent introduces two novel methods to correct for melanin absorption: (1) adding an additional LED wavelength around 600 nm that is absorbed by melanin but not by macular pigment, and (2) replacing the green LED in the traditional HFP setup with a red LED that is absorbed by melanin but not by macular pigment. These methods improve the accuracy of MPOD measurements, providing more reliable data for clinical and research applications.

### SUMMARY

The present invention relates to methods for measuring macular pigment optical density (MPOD) more accurately by accounting for melanin absorption. Traditional heterochromatic flicker photometry (HFP) techniques do not consider melanin, leading to inaccuracies. This patent introduces two novel methods to correct for melanin absorption: (1) adding an additional LED wavelength around 600 nm that is absorbed by melanin but not by macular pigment, and (2) replacing the green LED in the traditional HFP setup with a red LED that is absorbed by melanin but not by macular pigment. These methods improve the accuracy of MPOD measurements, providing more reliable data for clinical and research applications.

### FIELD OF THE INVENTION

The present invention relates to the field of ophthalmology and vision science, focusing on techniques for measuring macular pigment optical density (MPOD). Macular pigment is crucial for protecting the retina and enhancing visual performance. Accurate MPOD measurement is essential for assessing macular health and diagnosing conditions such as age-related macular degeneration (AMD). Traditional methods like heterochromatic flicker photometry (HFP) often overlook the absorption effects of melanin, a pigment in the retinal pigment epithelium and choroid, leading to inaccuracies. This invention introduces two novel methods to correct for melanin absorption, enhancing the precision and reliability of MPOD measurements. These methods have significant implications for clinical diagnostics, research, and treatment development related to macular health.

### BACKGROUND OF THE INVENTION

Macular pigment (MP), composed primarily of the carotenoids lutein and zeaxanthin and mezzo-zeaxanthin, is located in the macula of the human retina. MP plays a vital role in protecting the retina from oxidative damage and enhancing visual performance by filtering high-energy blue light. Measuring macular pigment optical density (MPOD) is crucial for evaluating macular health and assessing the risk of conditions such as age-related macular degeneration (AMD).

Heterochromatic flicker photometry (HFP) is a widely used technique for measuring MPOD. In HFP, a small circular stimulus alternates between two wavelengths: one absorbed by the macular pigment (typically blue at 460 nm) and one not absorbed (typically green at 540 nm). The subject adjusts the intensity of the absorbed wavelength until flicker is minimized or eliminated, both centrally and peripherally. The difference in intensity settings provides the MPOD value.

However, traditional HFP methods do not account for the presence of melanin, a pigment found in the retinal pigment epithelium and choroid, which also absorbs light, particularly in the blue and green regions of the spectrum. This absorption by melanin can interfere with accurate MPOD measurements, leading to potential inaccuracies in assessing macular health.

To address these limitations, this invention proposes two novel methods for improving the accuracy of MPOD measurements by accounting for melanin absorption:

1. Additional LED Wavelength: Introducing an LED with a wavelength around 600 nm, absorbed by melanin but not by the macular pigment, to measure melanin absorption directly and correct MPOD measurements accordingly.

2. Replacement of Green LED with Red LED: Replacing the green (540 nm) LED with a red LED (around 600 nm) in the traditional HFP setup, allowing for a simplified correction for melanin absorption.

By incorporating these methods, the accuracy and reliability of MPOD measurements are significantly enhanced, providing more precise data for clinical diagnostics, research, and treatment development related to macular health.

### SUMMARY OF THE INVENTION

The present invention addresses the limitations of traditional heterochromatic flicker photometry (HFP) methods in measuring macular pigment optical density (MPOD) by introducing corrections for melanin absorption. Melanin, a pigment found in the retinal pigment epithelium and choroid, can interfere with MPOD measurements, leading to inaccuracies. This invention proposes two novel methods to enhance the accuracy of MPOD readings:

### 1. Method 1: Additional LED Wavelength

Introduction: This method introduces an additional LED with a wavelength around 600 nm, which is absorbed by melanin but not by the macular pigment.
Instrumentation: The HFP setup is modified to include the additional LED alongside the traditional blue (460 nm) and green (540 nm) LEDs.
Calibration and Measurement Process: Measurements are taken using all three LEDs, allowing for the direct assessment of melanin absorption.
Correction Algorithm: An algorithm is developed to correct the MPOD readings by accounting for the melanin absorption data obtained from the 600 nm LED.
   2. Method 2: Replacement of Green LED with Red LED.
Introduction: This method replaces the traditional green (540 nm) LED with a red LED around 600 nm, which is absorbed by melanin but not by the macular pigment.
Instrumentation: The HFP setup is modified to use the blue (460 nm) and red (600 nm) LEDs instead of the blue and green combination.
Calibration and Measurement Process: The subject adjusts the intensity of the blue and red LEDs to minimize flicker, similar to the traditional HFP method.
Correction Algorithm: An algorithm is developed to correct the MPOD readings based on the absorption characteristics of melanin at the red wavelength.

By incorporating these methods, the present invention significantly enhances the accuracy and reliability of MPOD measurements. These improvements provide more precise data for clinical diagnostics, research, and the development of treatments related to macular health.

### BRIEF DESCRIPTION OF THE DRAWINGS

FIG. 1: Schematic of Traditional Heterochromatic Flicker Photometry (HFP) Setup. This figure illustrates the traditional HFP setup used for measuring macular pigment optical density (MPOD) using a single light source, lenses, mirrors, and filters.

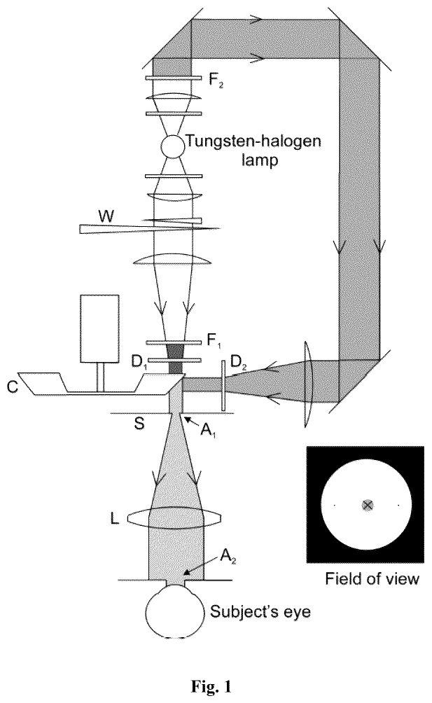

FIG. 2: Modified HFP Setup for Method 1 (Additional LED Wavelength). This figure shows the HFP setup modified to include an additional red LED (600 nm) for measuring melanin absorption, enhancing the accuracy of MPOD measurements.

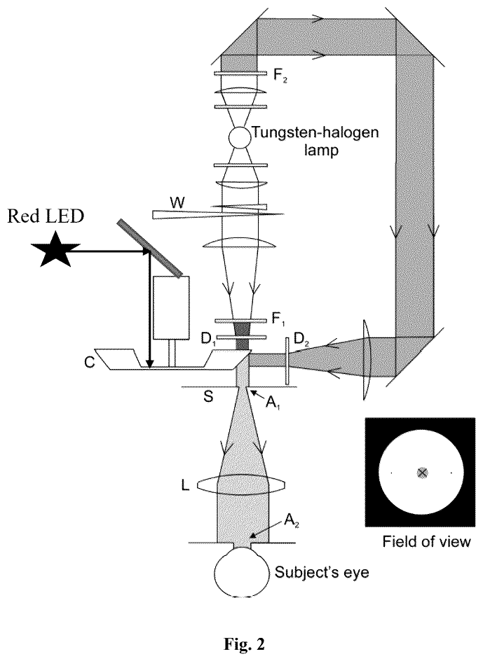

FIG. 3: Example Data and Correction Algorithm for Method 1 (Additional LED Wavelength). This graph presents hypothetical intensity settings and corrected MPOD calculations for Method 1, incorporating the additional LED to account for melanin absorption.

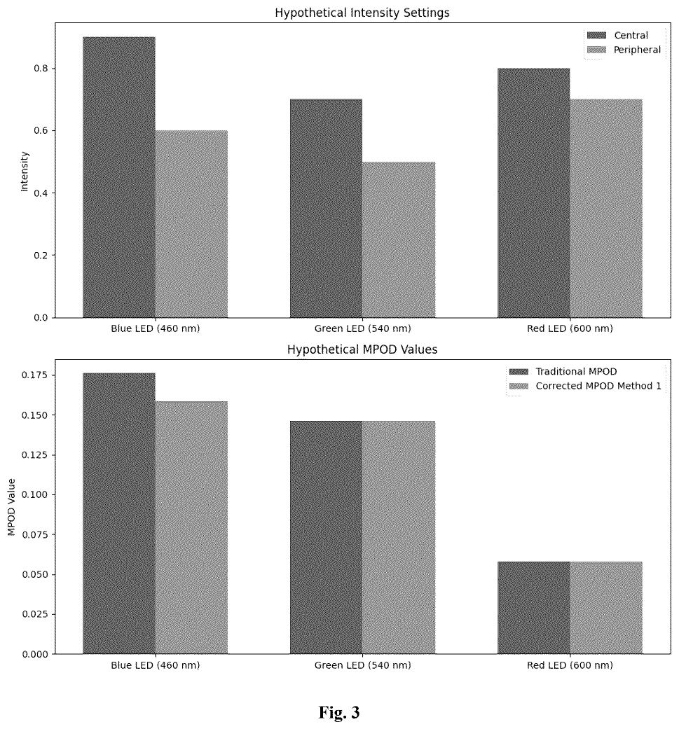

FIG. 4: Example Data and Correction Algorithm for Method 2 (Replacement of Green LED with Red LED). This graph displays the intensity settings and corrected MPOD calculations for Method 2, which replaces the green LED with a red LED for simplified correction for melanin absorption.

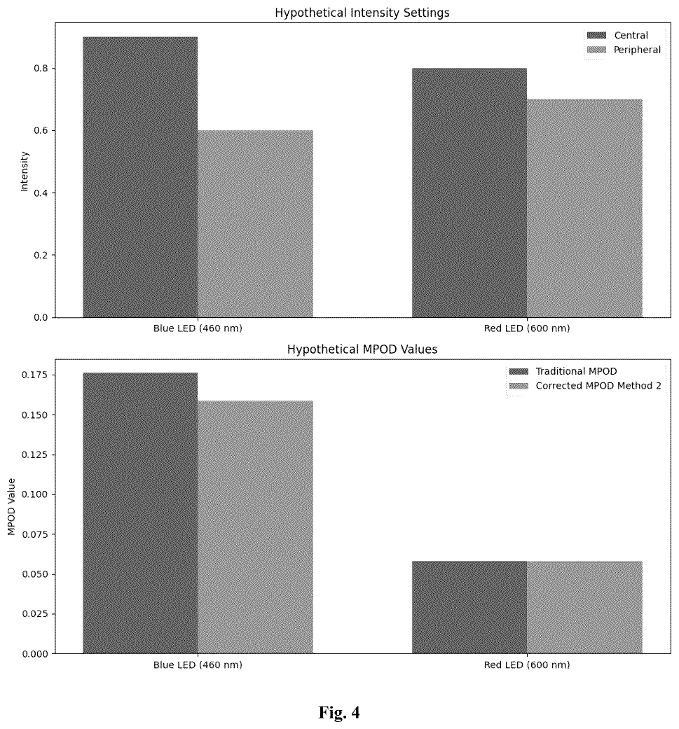

### DETAILED DESCRIPTION OF THE INVENTION

### Method 1: Additional LED Wavelength

Introduction: This method introduces an additional LED with a wavelength around 600 nm, which is absorbed by melanin but not by the macular pigment. This allows for a direct measurement of melanin absorption, improving the accuracy of MPOD measurements.

Instrumentation: The heterochromatic flicker photometry (HFP) setup is modified to include an additional LED with a wavelength of 600 nm. The setup now includes three LEDs:
Blue LED (460 nm) for MPOD measurement.
Green LED (540 nm) as a traditional reference.
Additional LED (600 nm) for melanin absorption measurement.

### Calibration and Measurement Process:

### 1. Calibration:

The device is calibrated using standard procedures to ensure the intensity of each LED is accurately measured.
Calibration involves adjusting the intensity of each LED to account for any baseline absorption and ensure consistent output.

### 2. Measurement:

Subjects view a small circular stimulus alternating between the blue (460 nm) and green (540 nm) LEDs.
The flicker observed by the subject is reduced to a null point by adjusting the intensity of the blue LED while viewing the stimulus centrally and peripherally.
The same process is repeated with the additional (600 nm) LED to measure melanin absorption.
The flicker observed by the subject is reduced to a null point by adjusting the intensity of the additional LED while viewing the stimulus centrally and peripherally.

### Correction Algorithm:

### 1. Mathematical Formulation:

The traditional MPOD measurement is calculated as:

$${MPOD}_{traditional} = log ( \frac{{I}_{central , blue}}{{I}_{peripheral , blue}} )$$

The melanin absorption at 600 nm is measured similarly:

$${Melanin}_{absorption} = log ( \frac{{I}_{central , red}}{{I}_{peripheral , red}} )$$

### 2. Algorithm Details:

The corrected MPOD is calculated by adjusting the traditional MPOD with the melanin absorption data:

$${MPOD}_{corrected} = {MPOD}_{traditional} - α \cdot {Melanin}_{absorption}$$

Here, a is a correction factor determined experimentally.
   Method 2: Replacement of Green LED with Red LED

Introduction: This method replaces the traditional green (540 nm) LED with a red LED around 600 nm, which is absorbed by melanin but not by the macular pigment. This simplifies the correction for melanin absorption.

Instrumentation: The HFP setup is modified to use the following LEDs:
Blue LED (460 nm) for MPOD measurement.
Red LED (600 nm) as a reference.

### Calibration and Measurement Process:

### 1. Calibration:

Similar to Method 1, the device is calibrated to ensure accurate intensity measurements for the blue and red LEDs.

### 2. Measurement:

Subjects view a small circular stimulus alternating between the blue (460 nm) and red (600 nm) LEDs.
The flicker is minimized by adjusting the intensity of the blue LED, both centrally and peripherally.

### Correction Algorithm:

### 1. Mathematical Formulation:

The traditional MPOD measurement is calculated as:

$${MPOD}_{traditional} = log ( \frac{{I}_{central , blue}}{{I}_{peripheral , blue}} )$$

The melanin absorption using the red LED is measured as:

$${Melanin}_{absorption} = log ( \frac{{I}_{central , red}}{{I}_{peripheral , red}} )$$

### 2. Algorithm Details:

The corrected MPOD is calculated by:

$${MPOD}_{corrected} = {MPOD}_{traditional} - β \cdot {Melanin}_{ab sorption}$$

Here, β is a correction factor determined through experimentation.

### EXAMPLES AND EXPERIMENTAL DATA

### Example 1: Theoretical Validation of Method 1 (Additional LED Wavelength)

Objective: To validate the potential accuracy of MPOD measurements using the additional red (600 nm) LED to account for melanin absorption.

### Hypothetical Scenario:

1. Participants: Consider a sample of 10 participants with varying levels of macular pigment and melanin content.
2. Instrumentation: Hypothetical HFP device with blue (460 nm), green (540 nm), and red (600 nm) LEDs.
3. Procedure:
   Calibrate the HFP device to ensure accurate intensity measurements for all three LEDs.
   Conduct hypothetical MPOD measurements using the blue and green LEDs, and record the intensity settings.
   Conduct hypothetical melanin absorption measurements using the red LED, and record the intensity settings.
   Apply the correction algorithm to adjust the MPOD measurements based on melanin absorption.

### Expected Results:

### 1. Raw Data:

Present hypothetical intensity settings for blue, green, and red LEDs for central and peripheral measurements.
Calculate the traditional MPOD using blue and green LED data.
Calculate melanin absorption using the red LED data.

### 2. Corrected MPOD:

Apply the correction factor (α\alphaα) to the traditional MPOD measurements.
Present hypothetical corrected MPOD values.

### 3. Comparison:

Compare the corrected MPOD values with those obtained using traditional methods without melanin correction.
Use theoretical statistical analysis to suggest the significance of the correction.

Discussion: The following points are intended to explain the significance of the hypothetical results to the reader:
Theoretical Improvements: By incorporating melanin absorption measurements, this method is expected to improve the accuracy of MPOD readings, which are critical for diagnosing and monitoring conditions like age-related macular degeneration (AMD).
Expected Trends: It is anticipated that the corrected MPOD values will show less variability and be more consistent across subjects with different levels of melanin, leading to more reliable data.
Practical Implications: The improvements in accuracy and reliability of MPOD measurements could enhance clinical diagnostics and research, potentially leading to better patient outcomes and advancements in understanding macular health.

### Example 2: Theoretical Validation of Method 2 (Replacement of Green LED with Red LED)

Objective: To validate the potential accuracy of MPOD measurements using the red (600 nm) LED as a replacement for the green (540 nm) LED to account for melanin absorption.

### Hypothetical Scenario:

1. Participants: Consider a sample of 10 participants with varying levels of macular pigment and melanin content.
2. Instrumentation: Hypothetical HFP device with blue (460 nm) and red (600 nm) LEDs.
3. Procedure:
   Calibrate the HFP device to ensure accurate intensity measurements for both LEDs.
   Conduct hypothetical MPOD measurements using the blue and red LEDs, and record the intensity settings.
   Apply the correction algorithm to adjust the MPOD measurements based on melanin absorption.

### Expected Results:

### 1. Raw Data:

Present hypothetical intensity settings for blue and red LEDs for central and peripheral measurements.
Calculate the traditional MPOD using blue and red LED data.
Calculate melanin absorption using the red LED data.

### 2. Corrected MPOD:

Apply the correction factor (β\betaβ) to the traditional MPOD measurements.
Present hypothetical corrected MPOD values.

### 3. Comparison:

Compare the corrected MPOD values with those obtained using traditional methods without melanin correction.
Use theoretical statistical analysis to suggest the significance of the correction.

Discussion: The following points are intended to explain the significance of the hypothetical results to the reader:
Theoretical Improvements: This method simplifies the correction process while still accounting for melanin absorption, leading to more accurate MPOD measurements.
Expected Trends: The use of the red LED is expected to provide consistent MPOD readings across different subjects, reducing variability and increasing reliability.
Practical Implications: Simplifying the measurement process makes the method more accessible for routine clinical use, potentially benefiting a larger number of patients and improving the quality of care.

1.:
Preamble: A method for measuring macular pigment optical density (MPOD) that accounts for melanin absorption.
Transitional Phrase: Comprising:
claim Body:
a. Using a heterochromatic flicker photometry (HFP) setup with blue (460 nm), green (540 nm), and red (600 nm) LEDs;
b. Calibrating the HFP device to ensure accurate intensity measurements for all LEDS;
c. Measuring MPOD by alternating between the blue and green LEDs and recording intensity settings;
d. Measuring melanin absorption by alternating between the blue and red LEDs and recording intensity settings;
e Calculating the traditional MPOD using the blue and green LED data;
f. Calculating melanin absorption using the red LED data;
g. Applying a correction algorithm to adjust the traditional MPOD measurements based on melanin absorption.

2.:
Preamble: A method for correcting melanin interference in optical density measurements.
Transitional Phrase: Comprising:
claim Body:
2. The method of claim 1, wherein the correction algorithm is defined as:
$${MPOD}_{corrected} = {MPOD}_{traditional} - α \cdot {Melanin}_{absorption}$$
where α is a correction factor determined experimentally.
Using absorbance values at two wavelengths, one that is absorbed by melanin and one that is not, to create a baseline for melanin correction;
Applying the baseline to correct the optical density measurements at a target wavelength affected by melanin absorption.

3.:
Preamble: A method for measuring macular pigment optical density (MPOD) that accounts for melanin absorption using a simplified setup.
Transitional Phrase: Comprising:
claim Body:
a. Using a heterochromatic flicker photometry (HFP) setup with blue (460 nm) and red (600 nm) LEDs;
b. Calibrating the HFP device to ensure accurate intensity measurements for both LEDs;
c. Measuring MPOD by alternating between the blue and red LEDs and recording intensity settings;
d. Calculating the traditional MPOD using the blue LED data;
e. Calculating melanin absorption using the red LED data;
f. Applying a correction algorithm to adjust the traditional MPOD measurements based on melanin absorption.
Dependent claims

4.:
The method of claim 3, wherein the correction algorithm is defined as:
$${MPOD}_{corrected} = {MPOD}_{traditional} - β \cdot {Melanin}_{absorption}$$
where β is a correction factor determined experimentally.
Preamble: The method of claim 1.
claim Body: Further comprising adjusting the correction algorithm based on experimental data to optimize the accuracy of the MPOD measurement.

5.:
a. A blue LED with a wavelength of 460 nm for MPOD measurement;
b. A green LED with a wavelength of 540 nm as a traditional reference;
c. A red LED with a wavelength of 600 nm for melanin absorption measurement;
d. A calibration system to ensure accurate intensity measurements for all LEDS;
e. A data processing unit to calculate traditional MPOD and melanin absorption;
f. A correction algorithm to adjust MPOD measurements based on melanin absorption.
Preamble: The method of claim 1.
claim Body: Further comprising standardizing the sampling area in optical measurements to ensure consistency across all subjects.

6.:
The device of claim 5, wherein the correction algorithm is defined as:
$${MPOD}_{corrected} = {MPOD}_{traditional} - α \cdot {Melanin}_{absorption}$$
where α is a correction factor determined experimentally.
Preamble: The method of claim 2.
claim Body: Further comprising applying the correction baseline to multiple target wavelengths within the spectral range affected by melanin absorption.

7.:
7. A heterochromatic flicker photometry (HFP) device for measuring macular pigment optical density (MPOD) that accounts for melanin absorption, comprising:
a. A blue LED with a wavelength of 460 nm for MPOD measurement;
b. A red LED with a wavelength of 600 nm for melanin absorption measurement;
c. A calibration system to ensure accurate intensity measurements for all LEDs;
d. A data processing unit to calculate traditional MPOD and melanin absorption;
e. A correction algorithm to adjust MPOD measurements based on melanin absorption.
Preamble: The method of claim 3.
claim Body: Further comprising adjusting the LED intensity based on subject-specific melanin levels detected during calibration.

8.:
The device of claim 7, wherein the correction algorithm is defined as:
$${MPOD}_{corrected} = {MPOD}_{traditional} - β \cdot {Melanin}_{absorption}$$
where β is a correction factor determined experimentally.
Preamble: The method of claim 3.
claim Body: Further comprising automating the calibration process to minimize user error and improve measurement consistency.
Independent claims (Device)

9.:
A method for improving the accuracy of macular pigment optical density (MPOD) measurements by incorporating melanin absorption data, comprising:
a. Using heterochromatic flicker photometry (HFP) with an additional wavelength that is absorbed by melanin but not by macular pigment;
b. Calibrating the HFP device for all wavelengths;
c. Measuring MPOD and melanin absorption;
d. Applying a correction algorithm to the MPOD data to account for melanin absorption.
Preamble: A heterochromatic flicker photometry (HFP) device for measuring macular pigment optical density (MPOD) that accounts for melanin absorption.
Transitional Phrase: Comprising:
claim Body:
A blue LED with a wavelength of 460 nm for MPOD measurement;
A green LED with a wavelength of 540 nm as a traditional reference;
A red LED with a wavelength of 600 nm for melanin absorption measurement;
A calibration system to ensure accurate intensity measurements for all LEDS;
A data processing unit to calculate traditional MPOD and melanin absorption;
A correction algorithm to adjust MPOD measurements based on melanin absorption.

10.:
The method of claim 9, wherein the additional wavelength is around 600 nm.
Preamble: A heterochromatic flicker photometry (HFP) device for measuring macular pigment optical density (MPOD) using a simplified setup.
Transitional Phrase: Comprising:
claim Body:
A blue LED with a wavelength of 460 nm for MPOD measurement;
A red LED with a wavelength of 600 nm for melanin absorption measurement;
A calibration system to ensure accurate intensity measurements for all LEDs;
A data processing unit to calculate traditional MPOD and melanin absorption;
A correction algorithm to adjust MPOD measurements based on melanin absorption.
Dependent claims (Device)

11.:
The method of claim 9, wherein the correction algorithm is adapted based on experimental data to optimize accuracy.
Preamble: The device of claim 9.
claim Body: Further comprising an automated calibration system that adjusts LED intensity based on detected melanin absorption levels.

12.:
The method of claim 9, wherein the calibration system includes procedures for adjusting LED intensity to ensure consistent output and accurate measurement.
Preamble: The device of claim 10.
Claim Body: Further comprising a user interface that displays real-time adjustments to the correction algorithm based on varying melanin absorption levels.
